(19) United States
(12) Patent Application Publication
(10) Pub. No.: US 2021/0182932 A1
(43) Pub. Date: Jun. 17, 2021

(54) Medical Travel Companion

(71) Applicant: Olaseni Akintola Bello, JR., Albany, CA (US)

(72) Inventor: Olaseni Akintola Bello, JR., Albany, CA (US)

(21) Appl. No.: 17/118,244

(22) Filed: Dec. 10, 2020

Related U.S. Application Data
(60) Provisional application No. 62/947,284, filed on Dec. 12, 2019.

(51) Int. Cl. G06Q 30/06; G16H 40/67; G16H 80/00

(52) U.S. Cl. CPC G06Q 30/0627; G16H 80/00; G16H 40/67

(57) ABSTRACT

A medical travel companion system that includes a user device that transmits an individual's medical information, insurance information, and travel information to a server. The server has a database and the database has at least one medical service provider information, which includes information about services each medical service provider offers and insurance policies each medical service provider accepts. The server compares the individual's medical information, insurance information, and travel information to the at least one medical service provider information to determine at least one medical service provider for providing a medical service to the individual. The server transmits the determined at least one medical service provider to the user device. The server translates the individual's medical information to a language that the determined at least one medical service provider can read.

### TECHNICAL FIELD

The disclosure herein relates to systems for identifying and locating qualified medical service providers in locations abroad. Travelers can face unexpected medical needs while traveling abroad and can face difficulties finding medical service providers that accept a traveler's medical insurance and can provide the type and quality of services a traveler may need. This is especially true when in a country they are not familiar with and where the traveler does not speak the native language. The disclosed system relates to identifying medical providers that are qualified and equipped to address a traveling individual's medical conditions while also able to accept a traveler's insurance. Further, the system relates to automatically providing medical records in a format and language useful to a selected medical service provider.

### BACKGROUND

Systems known in the art allow a user to find businesses in an area such as restaurants or bars based on search criteria. These criteria can include type of food or drink, price range, ambiance, type of service, or any other characteristic a patron considers when choosing a place to eat or drink. However, there are no systems available to a user that are designed and function to locate high-quality medical service providers, especially when traveling overseas. Unlike restaurants or bars, it can be difficult to find a suitable medical service provider for a user because a patient's needs are much more particular and critical when choosing a medical service provider than those that a patron considers when dining out. When searching for a medical service provider, selecting an unsuitable one can mean financial ruin, serious and permanent injury, or even death. When traveling in a foreign country, it is even more difficult to find a suitable and high-quality medical service provider due to unfamiliarity with the culture and area, and possibly a language barrier between the patient and the local population.

### SUMMARY

The needs set forth herein as well as further and other needs and advantages are addressed by the present embodiments, which illustrate solutions and advantages described below.

There is a need for a system that can search for medical service providers in foreign locations or those unfamiliar to the user. The system can take a number of factors into consideration including type of care needed, pre-existing medical conditions, insurance or health plan coverage, quality of care given, and care specialties. The system can provide recommendations for medical service that the user can trust.

Further, there is a need for the system to allow the user to provide medical information in an efficient and easily accessible format to the medical professionals because they may not speak the same language as the user. Accordingly, the system may translate and format the user's medical information such that the medical professionals at the medical service provider can read the medical information in their native language and in a format they are used to seeing. The system can provide useful and translated information to the medical professionals regarding the user's medical information.

One embodiment of the present teaching relates to a medical travel companion system having a user device transmitting a user medical information, insurance information, and travel information to a server. The server has a database and the database has a list of medical service providers and information about the services each medical provider offers. The database also has information regarding which insurance policies the medical provider accepts. The server accesses the database and compares the user medical information, insurance information, and travel information to the medical service providers' information to determine a medical service provider that can provide medical services to the individual, and that accepts the user insurance. The server transmits the determined medical service provider to the user device. The server translates the user medical information to a language that the medical service provider is able to read.

Another embodiment the present teachings relates to the medical travel companion system including a user device transmitting information identifying a second user device. A server associates the second user device with a safety companion status. The server transmits the user medical information to the second user device.

Another embodiment of the medical travel companion system includes an individual device transmitting a user medical service type preference information to a server. The server also compares the user medical service type preference information to medical service providers' information to determine the medical service provider that can provide medical services to the individual and that accepts the user insurance.

Another embodiment of the medical travel companion system comprises a user device transmitting an individual's medical information, insurance information, and travel information to a server; the server having a database, the database having at least one medical service provider information including information about services each medical service provider offers and insurance policies each medical service provider accepts; the server receiving a medical service provider search request from the user device, the server comparing the individual's medical information, insurance information, and travel information to the at least one medical service provider information to determine at least one medical service provider for providing a medical service to the individual, the server transmitting the determined at least one medical service provider to the user device.

Another embodiment of the medical travel companion system comprises the server translating the individual's medical information to a language that the determined at least one medical service provider is able to read.

Another embodiment of the medical travel companion system comprises the user device transmitting information identifying a second user device; the server associating the second user device with a safety companion status to the individual; the server transmitting the individual's medical information to the second user device.

Another embodiment of the medical travel companion system comprises emergency call information sent by the server to the user device to make an emergency call; and a message sent to at least one emergency contact on the user device.

Another embodiment of the medical travel companion system comprises a medical directive included in the individual's medical information that empowers decision making in case of emergency.

Another embodiment of the medical travel companion system comprises the at least one medical service provider being at least one of accepting the individual's insurance and being geographically close to the individual.

Another embodiment of the medical travel companion system comprises at least one of verification from a third party, a patient feedback, and a user review, included in the at least one medical service provider information.

Another embodiment of the medical travel companion system comprises the user device transmitting the individual's medical service preference information to the server; the server comparing the individual's medical service preference information to the at least one medical service provider information to determine the at least one medical service provider.

Another embodiment of the medical travel companion system comprises an authorization for transferring the individual's medical information sent from the user device to the server; the server transmitting the individual's medical information to a selected medical service provider based on a selection of the medical service provider from the determined at least one medical service provider.

Another embodiment of the medical travel companion system comprises the server transmitting a translated version of the individual's medical information to the selected medical service provider in a language that the selected medical service provider can read.

Another embodiment of the medical travel companion system comprises a computer; an electronic device in data communication with said computer; medical information associated with a user received by said computer in a first language; travel information associated with a user received by said computer; a database in data communication with said computer; medical service provider information stored in said database accessible to said computer; a query determined by said computer based on at least the medical information and travel information; a list of medical providers determined by said computer based at least in part on said query, each of the medical providers being associated with at least one language; the list of medical providers being displayed on the electronic device; a selection of a medical provider received by said computer; the computer providing a translation of the medical information from the first language to at least one language associated with medical provider to the medical provider.

Another embodiment of the medical travel companion system comprises information identifying a second user received by said computer, and the computer associating the second user with a safety companion status to the user and transmitting the user's medical information to the second user.

Another embodiment of the medical travel companion system comprises information that enables the electronic device to make an emergency call; and information that notifies the user's emergency contact.

Another embodiment of the medical travel companion system comprises a medical directive provided in the user's medical information that empowers decision making in case of emergency.

Another embodiment of the medical travel companion system comprises insurance information associated with the user received by said computer; wherein the query is determined by said computer based on at least the medical information, the travel information, and the insurance information.

Another embodiment of the medical travel companion system provides that at least one medical provider in the list of medical providers is geographically close to the user.

Another embodiment of the medical travel companion system provides that the medical service provider information includes information about services each medical provider offers and insurance policies each medical provider accepts.

Another embodiment of the medical travel companion system comprises medical service preference information associated with the user received by said computer, wherein the query is determined by said computer based on at least the medical information, the travel information, and the medical service preference information.

Another embodiment of the medical travel companion system comprises information that enables the electronic device to contact a transportation service to the medical provider.

Another embodiment of the medical travel companion system comprises an authorization for transferring the user's medical information received by said computer, wherein the computer transmits the user's medical information to the medical provider.

### BRIEF DESCRIPTION OF THE DRAWINGS

FIG. 1 is an embodiment of a system for organizing medical data and providing locations and information for healthcare providers.

FIG. 2 illustrates further functions of the embodiment of the system of FIG. 1 allowing a medical service provider to transmit information about itself to the server, populate the system's database, communicate with patients, and review patient reviews.

FIG. 3 illustrates user interface examples of the user devices illustrating some of the functions of the system of FIG. 1.

FIG. 4 illustrates a data pipeline example involving the server and the database of FIG. 1.

### DETAILED DESCRIPTION

The present teachings are described more fully hereinafter with reference to the accompanying drawings, in which the present embodiments are shown. The following description is presented for illustrative purposes only and the present teachings should not be limited to these embodiments. Any computer configuration and architecture satisfying the speed and interface requirements herein described may be suitable for implementing the system and method of the present embodiments.

In compliance with the statute, the present teachings have been described in language more or less specific as to structural and methodical features. It is to be understood, however, that the present teachings are not limited to the specific features shown and described, since the systems and methods herein disclosed comprise preferred forms of putting the present teachings into effect.

For purposes of explanation and not limitation, specific details are set forth such as particular architectures, interfaces, techniques, etc. in order to provide a thorough understanding. In other instances, detailed descriptions of well-known devices, circuits, and methods are omitted so as not to obscure the description with unnecessary detail.

The present teaching discloses a medical travel companion system built for national or international travel and providing a user (including patients and health providers) global access to the health data, allowing a person to travel without worrying about a possible or unforeseeable health issue regardless of whether the person is prepared or not. The medical travel companion system provides a solution when the travel insurance is unclear, unreliable, and falls short.

The medical travel companion system ensures national and international travel healthcare and on-demand medicine come together. Features of the medical travel companion system include, but are not limited to a function of “Electronic Health Data” which allows ownable, personalized, and translatable records to be globally recognized, a function of “Medical Equity” which provides curated and verified healthcare professionals across general medicine through specialty care, as well as patients feedbacks incorporated and applied to ensure comprehensive review, a function of “Medical Directive” which encourages and empowers decision making in case of emergency, as well as proxy of power of attorney to ensure traveler's wishes are adhered to and in a timely fashion, and a function of “Safety Travel Companion Mode” which allows that under exigent circumstances a previously chosen travel companion, who is also on the system, can access the patient's key data and relay it to a healthcare provider, and may also enables the patient's key data and situation to be relayed to pre-selected travel contacts. The function of “Safety Travel Companion Mode” may be also referenced as SOS feature. For example, the medical travel companion system may enable up to three travel contacts to be added to have access to the first individual's medical records and may allow up to two authorized healthcare agents to have access to the first individual's medical directive. The medical travel companion system engages a full network of healthcare professionals and providers to ensure health and wellness compromise is unnecessary.

The medical travel companion system allows a traveler to carry her or his health data and share while on the go with healthcare providers on a secure, multiple factor authentication, HIPAA compliant, platform. The medical travel companion system offers customers/patients immediate location of a healthcare provider wherever they are in the world and whether their insurance will be accepted. The medical travel companion system provides a Safety Travel Companion (STC) mode leveraging the location of users and their consent to have another trusted user provide their medical information during exigent circumstances. The medical travel companion system provides concierge service facilitating faster medical access. The medical travel companion system is capable of offering medical directives for the patient's peace of mind. The medical travel companion system allows the patients' medical records to be translated to the language of the patients' destination country upon arrival.

The medical travel companion system allows the user to obtain an app and create a user profile which includes the user's medically relevant information such as blood type, and allows the user to enter the travel destination, or itinerary, and the medical travel companion system is capable of showing the user the hospital that is best tailored to the user's medical needs and/or that accepts the user's insurance anywhere in the world.

The medical travel companion system also provides an early detection of outbreaks in a region, aids to search and rescue, and provides deployment of medical kits to a region.

The medical travel companion system provides a medical interface with machine learning algorithm, advanced imaging including 3D visualization and diagnostic imaging capability, a platform that promotes medical equity and safer travel, a Safety Travel Companion (STC) mode that allows your travel companion and/or travel contacts password-protected access to your medical records, concierge options for enhanced logistical support including transport, an app that directly interfaces with other services such as ride sharing apps, travel programs, airlines, access to health and wellness events on the itinerary, a mental health support while traveling, portability of health records, a option of Medical Directives, access to eastern and non-traditional medicine options, and a wellness section housed within the system that provides easy access to key medical advancement alerts based on the user's profile.

The medical travel companion system provides travelers with key medical essentials, prescription ordering, vaccine information, and outbreak warnings, streams travel insurance quotes on the system's website, offers the extended life care options on the concierge service, provides medical tourism facilitator, provides refugee and immigrant healthcare, provides company/business travel companion, provides study abroad healthcare access, provides travel security synced for the itinerary, and provides exclusive luxury travel and wellness experiences.

The medical travel companion system also provides the ability to incorporate transportation to the medical providers' offices or facilities within the app, for example, leveraging Uber, Lyft, or some other transportation medium's API within the application so users do not have to leave the app to get transportation.

The medical travel companion system also provides a medium for travelers to give their primary provider a “release of records” so the new provider, during travel, is able to receive key information. Additionally, the foreign provider is able to transmit “medical summaries” or “discharge records” to the primary. For example, consider situations where medications have different names in other parts of the world: Tylenol is called Panadol in Nigeria. The aim is to ensure consistency and safety.

The medical travel companion system provides a proprietary algorithm that generates optional number of recommendations for the system users, provides in-person and telehealth care for different languages requirement, provides immediately local emergency service calls and notification to the user's emergency contracts, makes the health records (including electronic health records) searchable such as by simple tap and select, provides an option of uploading or creating medial directives via six yes or no questions, provides updated travel guidelines, destination statues, facemask policy and traveler first detail regarding global or local health concern such as Covid-19.

In the present teaching, a person who is interested in finding a medical health provider while travelling is thereafter referred to as a medical seeker, and the medical health provider who can provide the medical health service to the medical seeker is thereafter referred to as a medical provider. Either a medical-seeker or a medical-provider can use a user device to communicate within a system of the present teaching and use the functions of the system. The user device may be an electronic device. An electronic device according to the present teaching may include at least one of, for example, a smart phone, a tablet personal computer (PC), a mobile phone, a video phone, an e-book reader, a desktop PC, a laptop PC, a netbook computer, a workstation, a server, a personal digital assistant (PDA), a portable multimedia player (PMP), an MP3 player, a mobile medical device, a camera, or a wearable device. The user device used by the medical seeker and the user device used by the medical provider may be different devices or the same device. The system may include hardware, such as a computer or a server, or components thereof. The system may provide environment in which a piece of software is executed, such as a web page, an application, or a remote desktop.

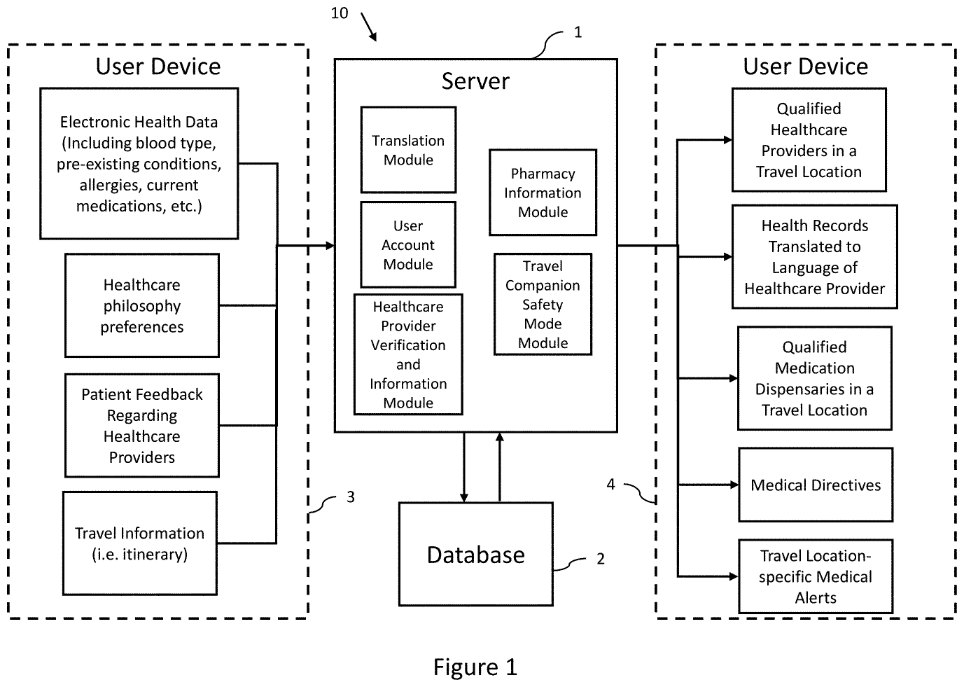

Referring to FIG. 1, in one embodiment, the system 10 can have a server 1 receiving medical and travel-related data from a user device 3 to create a user account for a medical seeker. For example, the medical seeker may enter their health data, including medical history of past surgeries, blood type, pre-existing conditions, allergies, and current medications, medical insurance policy data, and treatment philosophy into the user device 3. The user device 3 may transmit this information to the server 1 and the server 1 may process the information and store it in a database 2.

The user device 3 may request to search the qualified medical provider information based on a location. The user device 3 accessing the user account may transmit the request to the server 1 for the locations of medical service providers. Upon receiving the request from the user device 3, server 1 may search for the qualified medical providers based on the location information. The request may include search criteria including the previously entered data or additional search criteria including, but not limited to, location, additional medical service type, additional treatment philosophy, other insurance or health plans accepted as payment, relative cost of services, professional accreditation level, hours of operation, size of provider, or any other information known in the art to describe a medical service provider's operational scope and capacity. Alternatively, the request may only include the location information and in such case the server may use a preset or default search criteria to perform the search. Medical Provider recommendations may be generated from a methodology that leverages proprietary algorithms, machine learning processes, as well as destination and medical provider reference respective data decision trees.

The server 1 may then return a number of search results to the user device 3 or another user device 4 accessed by the medical seeker. The search result may be displayed on the user device 3 or 4 in human-readable format, including, but not limited to, a list, a table, or a graphical format. The graphical format may use global positioning system (GPS). The graphical format may include a map with icons showing the locations of medical service providers returned by the server 1. An individual icon may be included in the search result page showing the location of the user device 3 or 4, a remote travel location, or a location entered into the user device 3 or 4 as part of the search query. The map may include representations of streets, buildings, and geographical features. The icons may be placed on the map to show the location of the medical service provider in relation to any of the individual icons and other features on the map.

The icons may also represent information about the medical providers. The icons may include colors, shapes, sizes, or designs to represent the characteristics of the medical service provider. For example, a certain color icon may be used to indicate that the medical provider has a certified medical doctor on staff, or that they accept a certain medical insurance plan. Light or dark icons may be used to indicate whether the medical service provider is currently open.

The search result may also be output in an audio format.

The system 10 may allow an individual to order transportation from within the system's application. The system 10 may accept a request from a medical seeker to have a transporter arrive at a particular location to pick up the medical seeker, then transport the medical seeker to a selected medical provider. The system 10 may interface with external transportation services to order transportation including, but not limited to, taxi services, livery taxi services, Uber, Lyft, ambulance services, or any other service that provides transportation of individuals. The system 10's interface with an external transportation may include, but is not limited to, sending the location of a medical seeker, sending the location of a pickup spot, sending the location of a medical provider, sending the number of passengers to be picked up, and sending payment information.

Once a medical seeker finds a medical service provider that meets their needs, the system may further supply the medical service provider with vital medical information, especially when a medical seeker is in a foreign country and does not speak the native language enough to communicate technical or medical information. The system 10 may provide translated medical records and allow a medical seeker to provide them to a medical professional in a language and format that the medical professional understands. To do this, the user device 3 may transmit a request for translated records to the server 1. The request may include a selected language into which the system 10 translates the records. The server 1 may translate the medical records of the individual account to the language selected and transmit them to the user device 3 or 4. The user device 3 or 4 may then display them in a human-readable format or output them in an audio format on the user device 3 or 4. Alternatively, instead of transmitting the translated medical records to the user device 3 or 4, the user device 3 or 4 may request that the server 1 transmits the translated medical records directly to the selected medical service provider's computer system through email, secure medical records handling systems, or any other electronic communication method known in the art.

Alternatively, the user device 3 may transmit the identity information of a selected medical service provider to the system 10 and using this information, the system 10 may determine the language in which the records to be translated. The server may reference the database 2 that contains the medical service provider, make a correlation to the primary language preference of the selected medical service provider, and translate the medical records to that language.

As a further alternative, the system 10 may translate the medical information to the language of a country when the user device 3 arrives in the country. The system 10 may use the user device 3's GPS data, cell tower triangulation data, WIFI location data, IP address data, or any other data known in the art for locating a mobile device. As another alternative, the individual may input a travel schedule into the system 10 indicating the location of travel and time of arrival. The system 10 may translate the medical records to the travel location's language at the scheduled time of arrival, or based on the pre-identification of medical service providers.

As is often the case with medical emergencies, the patient may be incapacitated and unable to provide medical information to a medical service provider. The system 10 may allow a user to designate another user with another user account and allow the other user's access to the user's medical information. The system 10 can allow the other user's access to view, translate, and send the medical records as described above. The user may designate more than one other user to handle the user medical records and may revoke any designation at any time. The user may also specify the scope of permissions the other users have when accessing the user medical records including, but not limited to, viewing, translating, sending, and editing privileges. For example, an individual may designate their travel companion as having access to the user's medical information from the companion's user account. The user may then allow the companion to view and translate the user's medical records, but may choose to not allow the companion to electronically send the records to a medical provider. Further, the user may select which medical information the companion has access to and which the companion does not. This functionality can improve the security of the user sensitive medical information.

The system 10 may determine a quality rating for each medical service provider in its database 2. The rating may be determined by qualitative or quantitative measurement of factors including, but not limited to, individual reviews, trade group ratings, internet data aggregation, and government evaluation ratings. Other factors may originate from usage data in the system 10 including metrics showing how often an individual selects a medical provider, how often an individual revisits a medical service provider, how often a medical service provider is returned in a group of search results, and how long the medical service provider has been in the system's database 2. Administrators of the system 10 may also provide data for the system 10 to use in determining a rating including data from the administrator's interaction with the medical service provider such as questionnaire data and in-person evaluation data.

Medical information is sensitive and must be secured from unauthorized access by unauthorized parties. The system 10 may provide a secure database 2 and the devices of the system 10 may be connected through secured and encrypted data connections. The system 10 may offer multiple factor authentication access and may be HIPAA (Health Insurance Portability and Accountability Act of 1996) compliant.

The system 10 may include a software application (“application”) for communication between the devices of the system 10. For example, the user device 3 may have an application with a user interface. The application may allow an individual to create different user accounts depending on the type of functions an individual requires of the application. The types of user accounts may include, individual patient (“individual”), medical provider (“provider”), and administrative accounts. The type of user account may determine which functions of the application and system 10 are available to the user of the account.

A user account may provide functions useful to a traveler with current or potential medical needs. These functions may include, but are not limited to, organizing electronic health information, translating organized health information, providing data about verified healthcare providers, providing crowd sourced individual reviews of medical providers, a travel companion mode that allows an individual to designate an individual to control access to their medical and health data, and providing the location of qualified healthcare providers in a given area.

The application may organize health information by providing an area which an individual inputs medical information. Medical information may include preexisting conditions, current ailments, allergies, current and past medications, previous surgeries, age, weight, gender, race, or any other information known in the art related to the medical condition of an individual.

Health information may also include, but is not limited to, preferred medical treatment philosophy, preferred medical staff, and insurance and health payment plans. Medical treatment philosophy selections may include, but are not limited to whether an individual prefers a holistic or a western approach to medical treatment. Preferred medical staff selection may include whether an individual prefers to be treated by medical doctors, nurse practitioners, nurses, male staff, female staff, holistic health coaches, or any other medical care professional known in the art. Insurance and health payment plan selections may include medical insurance plans, HSAs, FSAs, cooperative medical plans, or any other medical payment plans known in the art. Alternatively, an individual may indicate to the system 10 that they have no medical payment plan and pay for medical care out of pocket.

The system 10 may also allow a user to upload medical documents to the system 10 from the user device 3 or from a location remote to the user device 3. The system 10 may also allow an individual to provide information about a remote location where the user medical documents are stored, whereby the system 10 may request the medical documents from the remote location. The system 10 may then receive the medical documents from the remote location. For example, an individual may have medical documents stored on the user device 3 and may upload them in PDF format. As a further example, the system 10 may allow the individual to send information about the individual's primary care doctor's office where the individual's medical documents are held. The system 10 may then send a request for medical documents directly to the primary care doctor with appropriate authorization documentation verifying the system is an authorized recipient of the individual's medical documents.

The system 10 may allow an individual to send their primary care doctor a “release of records” with which the individual authorizes the medical provider to receive medical documents and information from the primary care doctor. The medical documents and information may include key information in treating the individual.

The system 10 may also allow an individual to send the medical provider a “release of records” with which the individual authorizes the medical provider to transmit medical summaries or discharge records to the primary care doctor. These summaries or discharge records may include information regarding the services provided by the medical provider, conditions observed by the medical provider, and other medical information regarding the individual.

These records, summaries, and discharge records may be translated by the system 10 to a language understood by the receiving party to ensure consistency and safety. The system 10 may also identify medication names from a region and substitute them for the name of the same medication of the region where the record recipient is located.

Once medical data is entered, the application and system 10 may send the data to the server 1 where a user account module may organize the medical data into a useful format. Once organized, the server 1 may send the organized data to a database 2 for storage. The server 1 and user account module may retrieve or edit the organized medical data in the database 2 either automatically or at the direction of a user.

The system 10 may provide translated medical data at the direction of an individual. A user device 3 may transmit an instruction to the server 1 to provide translated medical data. Upon receipt of said instruction, the translation module in the server 1 may retrieve the organized medical data and translate the organized medical data to a different language, including the native language of an area. The different language may be chosen manually by the individual or automatically by the system 10. The system 10 may choose the different language by referencing data including a preference input by the individual at an earlier time, location data obtained from the user device 3, location information regarding a healthcare provider, or any other data known in the art for determining a useful, preferred, or native language.

The system 10 may allow an individual to select a second individual to control access to the individual's medical data. This function may be called a “Travel Companion Safety Mode.” This mode may allow a first individual to select a second individual who also has an individual account to access the first individual's medical records in the event the first individual is incapacitated and needs medical services. The second individual may access the first individual's medical data from the second individual's individual account and handle the first individual's medical information in the same manner that the first individual would from their individual account. This may include viewing the information, editing the information, and sending the information to a medical service provider. The second individual may also direct the system 10 to translate the medical data to a different language.

The system 10 may have a list of qualified healthcare providers on a database 2 connected to the server 1. The list of healthcare providers may be maintained by the healthcare provider verification and information module. An individual having a provider account may input information into a user device 3 that may in turn transmit the information to the server 1. The healthcare provider verification and information module may then transmit the healthcare provider information to the database 2 for storage.

The system 10 may use the location of the requesting mobile device to determine the location of the area in which to search for a medical service provider. The system 10 may also use the location of the requesting mobile device to determine the size of the search area by taking into consideration the building density of the area in which the device is located. For example, the system 10 may search for medical providers within a smaller radius in an urban environment while it can search in a larger radius in a rural environment.

The system 10 may determine the order in which healthcare provider search results are displayed based on factors including proximity to the user device 3, user rating, system rating, qualification, medical expertise, size of provider, hours provider operates, price, insurance they will accept, treatment philosophy, or any other factor known in the art that patients use to select a healthcare provider. The system 10 may also flag healthcare providers as “recommended” or another similar status to indicate that they are particularly well-matched to the user search query and medical needs. For example, the system 10 may list one healthcare provider higher than another because, all other factors being equal, one of the providers accepts the user's insurance while the other does not. Further, one could be flagged as “recommended” because it has a particularly high user review rating.

The system 10 may also use the subject matter of the medical records as a factor in determining medical service provider search results. The system may use natural language processing, word identification, or any other method known in the art for interpreting documents to determine the information contained in the medical records. The system 10 may then compare the subject matter of the medical records to the specialties, capabilities, equipment, or any other characteristic of a medical service provider to determine if a medical service provider should be included in a set of search results. For example, the system 10 may identify pre-existing conditions and use said conditions as a factor in determining whether a medical service provider is capable of effectively treating a person with said pre-existing condition and should be included in the search results.

The system 10 may also use the interpreted information from the medical records to infer that an individual has, or likely has, another condition or complication not noted in the medical records. The system may use machine learning, pre-programed application, database, or any other method known in the art for automatically creating an inference from a data set. The system 10 may then use this inference as a factor in determining whether a medical service provider should be included in the search results.

The system 10 may allow the individual to enter additional information about their current symptoms and include this information in the search query for medical service providers. Using the current symptom information, the system 10 may determine what medical condition the individual has, or likely has, and use this information to determine whether a medical service provider should be included in the search results. The system 10 may use machine learning, a pre-programmed application, a database, or any other method known in the art for automatically creating an inference from a data set to determine what medical condition the individual has, or likely has.

The system 10 may compare the inferences deduced from the medical records and compare them to the specialties, capabilities, equipment, or any other characteristic of a medical service provider to determine if a medical service provider should be included in a set of search results.

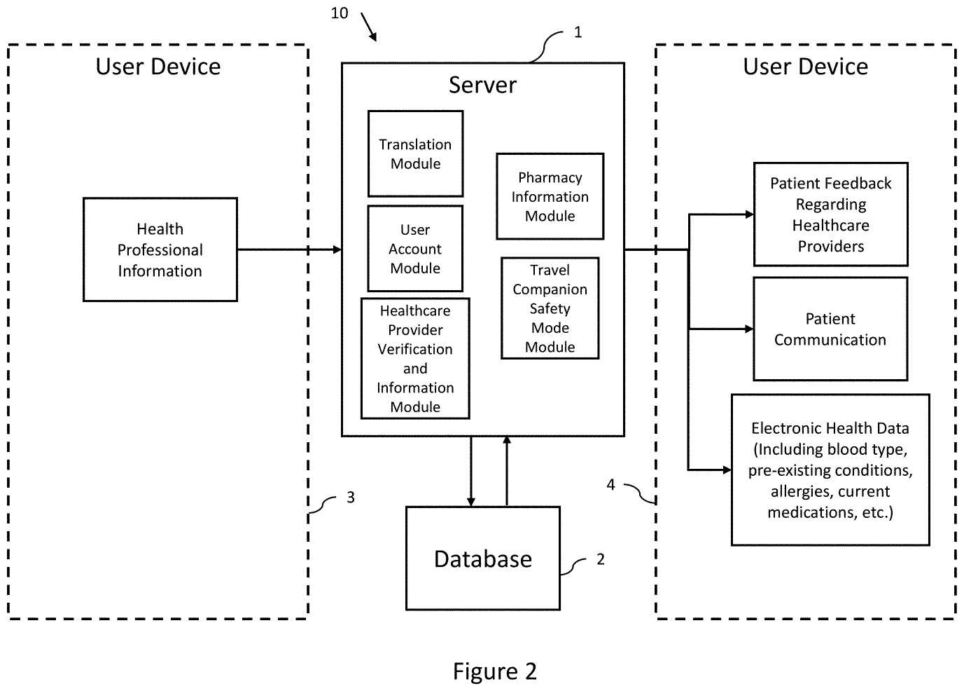

Referring to FIG. 2, the system 10 may have the server 1 receiving medical provider data from a user device 3 to create a medical service provider user account (“provider account”) for a medical provider. A medical provider may use the provider account to register their organization with the system 10 for potential medical seeker to find. Information that a medical provider may input to the system 10 includes, but is not limited to, location of the provider, types of medical services offered, acceptance of insurance or health plans as payment, payment methods accepted, estimated cost of services, professional accreditation level, hours of operation, size of provider, staff metrics, treatment philosophy, treatment equipment, directions to the provider's organization, and any other characteristic known in the art for describing a medical service provider to the public. The system 10 may allow the medical provider to update information describing their organization. Updates may be automatically processed by the system 10 or be subject to authorization by a system administrator.

The system 10 may allow a medical provider to communicate with a potential patient through the system 10 and user device 3 or 4 through text, audio, or video messaging. This communication may allow a medical provider to aid a potential patient in obtaining directions to a medical provider, determine if a medical provider may treat a potential patient, instruct a potential patient on how to manage their medical situation until they reach a provider, or to communicate any other information known in the art for assisting a potential patient.

The system 10 may allow a medical provider to propose edits to the medical information of an individual. The medical provider may use the provider account to send proposed information edits to the server 1. Subsequently, the server 1 may send an authorization prompt to the user account that owns the information the medical provider proposes to edit. The user may then accept or deny the change to the medical information. If the proposed edit is accepted, the server 1 may edit the medical information stored in the database 2. If the proposed change is denied, then no change is made to the medical information. This feature allows medical information to be updated so the user has accurate information throughout their travels abroad, but also secures the user information against unauthorized or inaccurate changes.

The medical provider may also review user reviews of their organization. When a user who has used the services of the provider enters a review into the system 10, the medical provider may be notified by the system 10 that a new review has been entered. The system 10 may provide the medical provider with the option to comment on, agree with, or protest a user review. An administrator of the system 10 may then be alerted by the system 10 that a medical provider has given input on a user review and take appropriate actions under operating protocols. The system 10 may process the user review automatically upon acceptance from the medical provider and flag the user review for administrative review upon protest from the medical provider.

For example, a first individual may use a mobile smart phone to open the system's application on the smart phone. The first individual may open an individual account and input their medical information. The information input may include age, weight, height, race, that they have type 2 diabetes, and that they are on a controlling medication. The first individual then may input that their insurance company “Star Insurance” provides medical payments under policy number 1005. The first individual further may upload medical documents from their primary care physician in the United States of America. The system 10 may identify that the U.S. is the first individual's home country because the primary care doctor is in the U.S.

The smart phone transmits the above information to the server 1 for processing and storage. The server 1 establishes an individual account and associates the input information with the account. The server 1 then stores the information on a hard disk drive as a database 2.

The first individual then may designate a second individual having a second individual account as having access to the first individual's medical records. The second individual may be a travel companion and may be allowed to view the translated medical records of the first individual. The second individual may also send the translated records to a medical service provider through email. The first individual may allow the second individual this access through the system 10 application and their own individual account. The first individual may choose to not allow the second individual to edit medical information from the second individual account.

The first individual travels to Madrid and, upon entering Spain, the system 10 may detect that the first individual's smart phone has entered Spain. The server of the system 10 may then translate the first individual's medical records to European Spanish, the native language of Spain, and may format the medical data in a way that is common to medical professionals in Spain.

While traveling, the first individual experiences a diabetic emergency and needs professional medical help. The first individual may input a query to the system's application through their smart phone. The system 10 may return 15 matches to the first individual's query and display them on the user device 3. The user device 3 may display a map of the first individual's area showing 15 medical providers within the city of Madrid. The first individual may identify an icon representing a medical provider that accepts the first individual's insurance and is currently open for treating patients. The first individual may select that provider and the system may contact the provider and inform them that the first individual is on their way to the provider for treatment of a diabetic emergency.

On the way to the provider, the first individual falls unconscious but arrives nonetheless. The second individual then, through their individual account, may provide the first individual's translated medical information to the provider. The first individual is treated and recovers.

The first individual, upon leaving the provider, may use the system 10 to give the provider a positive review for the care the first individual received. The provider may be notified of this review through their provider account. The provider may read the review and accept the review through the system 10. The system 10 then may use the positive individual review to calculate a quality of care rating in the system 10. This quality of care rating may be a factor in the ranking of the provider in subsequent search queries.

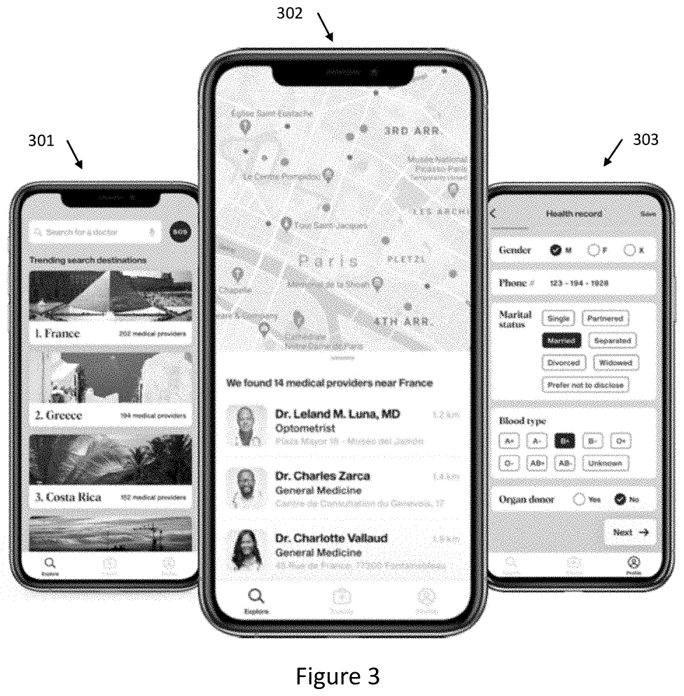

Regarding to FIG. 3, the left screen 301 illustrates a user interface for a search of medical providers based on locations. The middle screen 302 illustrates a user interface for a search result of medical providers. The right screen 303 illustrates a user interface for health record input by a user or received from third-party resources.

The system may provide health providers recommendations or a search result of health providers by various approaches. To ensure the quality of the prospective health providers, the system may conduct a “hard data” portion of data processing based on data provided by health providers themselves or third-party data sources. Additionally, the system may conduct a “soft data” portion of data processing based on, for example, the extensive local and human review that takes place to parse, assess, evaluate, and make recommendations.

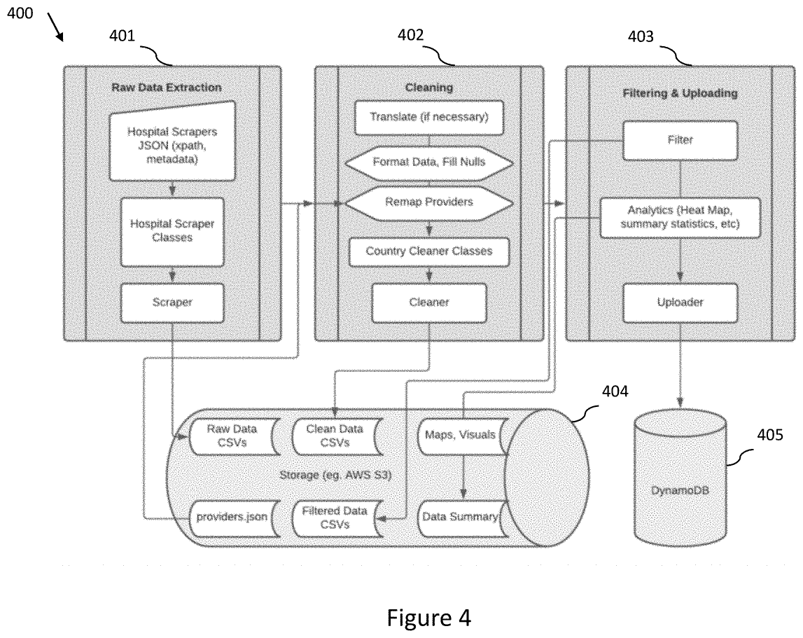

An exemplary hard data approach is shown in FIG. 4. A data pipeline 400 for securing health providers from first and third party data sources in the system of FIG.1 is illustrated. The data regarding the health providers may be provided by the health providers themselves or third-party data sources. The data pipeline 400 may include raw data extraction 401, cleaning 402, and filtering and uploading 403.

Raw data extraction 401 may include extracting raw data using different methods and/or different criteria and providing the extracted data. For example, hospital websites may be scraped for relevant data. The extracted data base be stored in a spreadsheet, CSV, or database on a storage 404.

Cleaning 402 may include, but not limited to, translating, formatting, filling nulls, and providing the clean data. Remapping of the data may be performed based on pre-defined providers. The cleaned data may be stored in a spreadsheet, CSV, or database on a storage 404.

Filtering and uploading 403 may include, but not limited to, filtering data using different methods and/or different criteria. Analytics may be performed on the data, such as to generate a heat map or summary statistics. The filtered data may be stored in a spreadsheet, CSV, or database on a storage 404. Maps, visuals and data summaries may also be stored on a storage 404. The filtered data may then be uploaded to a database 405 such as DynamoDB.

The quality of the health providers is one of the influential criteria that the system focuses upon. The search engines provided in the system may also use objectively criteria-based search approaches that may deploy quality as a complementary filter or a primary filter. The search result of the health providers may include a “matching/comparison” mechanic so that it reflects objective result verse subjective selection, and may be not just profile based, but medical need based.

While the present teachings have been described above in terms of specific embodiments, it is to be understood that they are not limited to these disclosed embodiments but can include any combination of the features of the described embodiments. Many modifications and other embodiments will come to mind to those skilled in the art to which this pertains, and which are intended to be and are covered by this disclosure. It is intended that the scope of the present teachings should be determined by proper interpretation of the disclosure and its legal equivalents, as understood by those of skill in the art relying upon this specification and the attached drawings.

Generally, all terms used in the claims are to be interpreted according to their ordinary meaning in the technical field, unless explicitly defined otherwise herein. All references to a/an/the element, apparatus, component, means, step, etc. are to be interpreted openly as referring to at least one instance of the element, apparatus, component, means, step, etc., unless explicitly stated otherwise. The steps of any method disclosed herein do not have to be performed in the exact order disclosed, unless explicitly stated. The use of “first”, “second,” etc. for different features/components of the present disclosure are only intended to distinguish the features/components from other similar features/components and not to impart any order or hierarchy to the features/components.

To aid the Patent Office and any readers of any patent issued on this application in interpreting the claims appended hereto, Applicant that it does not intend any of the appended claims or claim elements to invoke 35 U.S.C. 112(f) unless the words “means for” or “step for” are explicitly used in the particular claim.

What is claimed is:

1. A medical travel companion system comprising:
a user device transmitting an individual's medical information, insurance information, and travel information to a server;
the server having a database, the database having at least one medical service provider information including information about services each medical service provider offers and insurance policies each medical service provider accepts;
the server receiving a medical service provider search request from the user device, the server comparing the individual's medical information, insurance information, and travel information to the at least one medical service provider information to determine at least one medical service provider for providing a medical service to the individual, the server transmitting the determined at least one medical service provider to the user device.

2. The medical travel companion system of claim 1, further comprising the server translating the individual's medical information to a language that the determined at least one medical service provider is able to read.

3. The medical travel companion system of claim 1, further comprising:
the user device transmitting information identifying a second user device;
the server associating the second user device with a safety companion status to the individual;
the server transmitting the individual's medical information to the second user device.

4. The medical travel companion system of claim 1, further comprising:
emergency call information sent by the server to the user device to make an emergency call; and
a message sent to at least one emergency contact on the user device.

5. The medical travel companion system of claim 1, further comprising a medical directive included in the individual's medical information that empowers decision making in case of emergency.

6. The medical travel companion system of claim 1, further comprising the at least one medical service provider being at least one of accepting the individual's insurance and being geographically close to the individual.

7. The medical travel companion system of claim 1, further comprising at least one of verification from a third party, a patient feedback, and a user review, included in the at least one medical service provider information.

8. The medical travel companion system of claim 1, further comprising:
the user device transmitting the individual's medical service preference information to the server;
the server comparing the individual's medical service preference information to the at least one medical service provider information to determine the at least one medical service provider.

9. The medical travel companion system of claim 1, further comprising:
an authorization for transferring the individual's medical information sent from the user device to the server;
the server transmitting the individual's medical information to a selected medical service provider based on a selection of the medical service provider from the determined at least one medical service provider.

10. The medical travel companion system of claim 9, further comprising the server transmitting a translated version of the individual's medical information to the selected medical service provider in a language that the selected medical service provider can read.

11. A medical travel companion system, comprising:
a computer;
an electronic device in data communication with said computer;
medical information associated with a user received by said computer in a first language;
travel information associated with a user received by said computer;
a database in data communication with said computer;
medical service provider information stored in said database accessible to said computer;
a query determined by said computer based on at least the medical information and travel information;
a list of medical providers determined by said computer based at least in part on said query, each of the medical providers being associated with at least one language;
the list of medical providers being displayed on the electronic device;
a selection of a medical provider received by said computer;
the computer providing a translation of the medical information from the first language to at least one language associated with medical provider to the medical provider.

12. The medical travel companion system of claim 11, further comprising:
information identifying a second user received by said computer, and
the computer associating the second user with a safety companion status to the user and transmitting the user's medical information to the second user.

13. The medical travel companion system of claim 11, further comprising:
information that enables the electronic device to make an emergency call; and
information that notifies the user's emergency contact.

14. The medical travel companion system of claim 11, further comprising a medical directive provided in the user's medical information that empowers decision making in case of emergency.

15. The medical travel companion system of claim 11, further comprising insurance information associated with the user received by said computer; wherein the query is determined by said computer based on at least the medical information, the travel information, and the insurance information.

16. The medical travel companion system of claim 11, wherein at least one medical provider in the list of medical providers is geographically close to the user.

17. The medical travel companion system of claim 11, wherein the medical service provider information includes information about services each medical provider offers and insurance policies each medical provider accepts.

18. The medical travel companion system of claim 11, further comprising medical service preference information associated with the user received by said computer, wherein the query is determined by said computer based on at least the medical information, the travel information, and the medical service preference information.

19. The medical travel companion system of claim 11, further comprising information that enables the electronic device to contact a transportation service to the medical provider.

20. The medical travel companion system of claim 11, further comprising an authorization for transferring the user's medical information received by said computer, wherein the computer transmits the user's medical information to the medical provider.
